(19) United States
(12) Patent Application Publication
(10) Pub. No.: US 2026/0294367 A1
(43) Pub. Date: Oct. 1, 2026

(54) COMPUTED TOMOGRAPHY FACILITY WITH ABSORBER RINGS

(71) Applicant: Siemens Healthineers AG, Forchheim (DE)

(72) Inventors: Joerg FREUDENBERGER, Kalchreuth (DE); Martin BRAEUER, Nuernberg (DE); Christian HOFMANN, Erlangen (DE)

(73) Assignee: Siemens Healthineers AG, Forchheim (DE)

(21) Appl. No.: 19/576,470

(22) Filed: Mar. 24, 2026

(30) Foreign Application Priority Data
Mar. 25, 2025 (DE) 10 2025 111 493.5

(51) Int. Cl. A61B 6/42; A61B 6/03; A61B 6/40

(52) U.S. Cl. CPC A61B 6/4291; A61B 6/03; A61B 6/4007; A61B 6/4275

(57) ABSTRACT

A computed tomography device includes a gantry having an X-ray source unit, an X-ray detector unit and a tunnel. The X-ray source unit is arranged longitudinally offset relative to the X-ray detector unit with respect to a longitudinal direction of the tunnel. The X-ray source unit has X-ray source points arranged in a ring-shaped manner about the tunnel and in at least one X-ray emitter, which has a fixed position with respect to the gantry. The X-ray detector unit has a detector surface embodied in a ring-shaped manner about the tunnel and has a fixed position with respect to the gantry. The X-ray detector unit has a scatter radiation shield, which has absorber elements made from X-ray-absorbing material. The absorber elements are embodied as absorber rings arranged in a ring-shaped manner about the tunnel and has a fixed position with respect to the gantry.

### CROSS-REFERENCE TO RELATED APPLICATION(S)

The present application claims priority under 35 U.S.C. § 119 to German Patent Application No. 10 2025 111 493.5, filed Mar. 25, 2025, the entire contents of which are incorporated herein by reference.

### FIELD

One or more example embodiments of the present invention relate to a computed tomography facility (also referred to herein as a computed tomography device).

### BACKGROUND

A computed tomography facility is embodied in particular for computed tomography. Computed tomography is a very important method for achieving three-dimensional X-ray image data sets. In the prior art, this usually involves a rotating assembly in which the following apparatuses are arranged permanently connected to one another: an X-ray source unit, a collimator, a scatter radiation shield, and an X-ray detector unit. Also associated therewith in particular are the elements which control or supply said apparatuses, such as for example a high-voltage generator, an image data preprocessing computer, an emitter cooling system, and so on.

An increase in the time resolution of the detectable X-ray image data sets is usually limited by the centrifugal forces exerted while the assembly is rotating. Efforts are therefore being made to achieve three-dimensional X-ray image data sets without rotation, for example in the form of what are known as static computed tomography facilities or non-mechanical computed tomography facilities and/or by means of multi-emitter X-ray sources.

A challenge in computed tomography is the efficient suppression of X-ray scatter radiation. X-ray scatter radiation is understood to mean photons which do not reach a detector element of the X-ray detector unit via a direct path from an X-ray source point of the X-ray source unit but instead are scattered once or several times along the way. Such photons regularly disrupt the reconstruction of the X-ray image data sets, usually impairing quality, and are therefore to be suppressed as efficiently as possible.

In conventional computed tomography with a rotating X-ray source unit, X-ray scatter radiation is typically filtered out through grid-like structures made from X-ray-absorbing material. Even older are structures which extend exclusively in the longitudinal direction of the tunnel. All these structures typically have rectangular, in particular square, openings. A size of the opening of a grid-like structure typically correlates or corresponds to the size of a detector pixel in the order of magnitude of 1 mm. Here, the height of the structure is approximately 20 mm. The shaft thus produced is typically aligned precisely with the ring on which the rotating X-ray source unit moves. As a result, each opening of the grid-like structure is therefore typically aligned with an X-ray source point of the X-ray source unit. Such a design is however disadvantageous for static computed tomography facilities with several X-ray emitters having a fixed position.

### SUMMARY

An object underlying one or more example embodiments of the present invention is to specify an improved computed tomography facility (also referred to herein as a computed tomography device) in which in particular the X-ray scatter radiation is suppressed efficiently.

One or more example embodiments of the present invention relate to a computed tomography facility (also referred to herein as a computed tomography device) having a gantry,
wherein the gantry has an X-ray source unit, an X-ray detector unit and a tunnel,
wherein the X-ray source unit is arranged longitudinally offset relative to the X-ray detector unit with respect to the longitudinal direction of the tunnel,
wherein the X-ray source unit has several X-ray source points, which are arranged in a ring-shaped manner about the tunnel and in at least one X-ray emitter which has a fixed position with respect to the gantry,
wherein the X-ray detector unit has a detector surface, which is embodied in a ring-shaped manner about the tunnel and has a fixed position with respect to the gantry,
wherein the X-ray detector unit furthermore has a scatter radiation shield,
wherein the scatter radiation shield has several absorber elements made from X-ray-absorbing material, characterized in that
the several absorber elements are embodied exclusively as absorber rings, which are arranged in a ring-shaped manner about the tunnel and have a fixed position with respect to the gantry.

In the present application, in a ring-shaped manner or ring means that a length of the circular arc of the ring is greater than 180° and at most equal to 360°. In the latter case, the ring is a full circle. The length of the circular arc is at least greater than 180° because, in computed tomography, a representation of a slice during imaging typically requires the slice to be transilluminated with greater than 180° coverage. It is also conceivable for the circular arc to have a length of 225° or 270° or 315° or intermediate values of the afore-cited examples. In the following, the circular arc length of a ring thus designates the entire extent of the full circle or also lengths of more than 180°, depending on the embodiment. Here, the circular arc length of all subsequent rings and/or ring-shaped arrangements is equally long. The radius of the ring is typically at least 10 cm and/or at most 100 cm.

The computed tomography facility is in particular a static computed tomography facility, since neither the at least one X-ray emitter nor the detector surface are equipped to be rotatable with respect to the gantry. The computed tomography facility is embodied in particular for diagnostic imaging, for example computed tomography. Alternatively or additionally, the computed tomography facility can be embodied for technical imaging, for example material testing, non-destructive testing, customs checks and/or baggage checks.

The gantry of the computed tomography facility is a ring-shaped structure, which has the main components of the computed tomography facility and surrounds the tunnel, usually completely. Said components comprise the X-ray source unit, which generates the X-rays for imaging, and an X-ray detector unit containing the detector surface, which can detect the X-rays passing through the body of the patient or the object. The gantry is constructed so as to hold these components in a fixed position with respect to the tunnel. This enables precise imaging without the mechanical challenges of a rotating assembly. The tunnel is usually dimensioned such that for example an object and/or, via a patient couch, a patient can be arranged within the gantry so that the imaging can be carried out via the computed tomography facility.

Arranged longitudinally offset in the longitudinal direction means in particular arranged one behind the other. The X-ray source unit and the X-ray detector unit are therefore arranged one behind the other with respect to the longitudinal direction of the tunnel. This is typically required because the X-ray source points arranged in a ring-shaped manner and the detector surface embodied in a ring-shaped manner should have several viewing axes relative to one another. In other words, X-rays emanating from any X-ray source point must be able to strike ideally at least one detector pixel, typically several detector pixels, of the detector surface for the imaging. The arrangement one behind the other can usually ensure that this condition is met. It is preferable for the distance between these two rings in the longitudinal direction of the tunnel to be as short as possible.

In the present application, an X-ray emitter is defined by the fact that the X-ray emitter has a vacuum housing which can be closed in a vacuum-tight manner. Two X-ray emitters each have a vacuum housing which can be closed in a vacuum-tight manner, so that for example there are two vacuum housings each having a structurally separate vacuum, and so on. Here, no distinction is made between the terms X-ray emitter and X-ray tube.

It is possible in principle for X-rays to be emitted in the direction of the detector surface, simultaneously or consecutively, from each of the several X-ray source points. For this purpose, electrons are accelerated from a cathode in the direction of an anode within a high vacuum by a high voltage, which typically lies below 200 kV. The high voltage can lie in particular between 40 kV and 150 kV. The high vacuum is maintained in particular by the respective vacuum housing. The electrons are generated at the cathode by an electron emitter. The electrons can be generated by one or several electron emitters. The at least one electron emitter can be for example a thermionic filament emitter or flat emitter. Alternatively, the at least one electron emitter can be a field-effect emitter. The at least one anode can be embodied as a rotary anode or stationary anode. The accelerated electrons interact with the anode in what is known as the focal spot in order to generate X-rays there. Such a focal spot by definition forms an X-ray source point. A magnetic and/or electrical electron deflection facility can advantageously move the electrons back and forth between different focal spots.

It is in principle possible for the several X-ray source points to be arranged in just a single X-ray emitter. In this case, the single X-ray emitter is embodied to be ring-shaped and to have a fixed position, and surrounds the tunnel. The several X-ray sources are usually distributed over several X-ray emitters. In this case, the several X-ray emitters are arranged in a ring-shaped manner about the tunnel and in each case have a fixed position. Here, each X-ray emitter can comprise more than one X-ray source point. It is conceivable for each X-ray emitter to comprise just one X-ray source point.

The detector surface is formed in particular by several detector cell rows of the X-ray detector unit. The X-ray detector unit comprises in particular the several detector cell rows. The several detector cell rows are in particular arranged one behind the other with respect to the tunnel. The several detector cell rows are arranged as closely together as possible in order to avoid blind spots within the detector surface. The detector surface is usually pixelated in the longitudinal direction of the tunnel and in the circumferential direction of the tunnel. The detector surface of the X-ray detector unit is in particular aligned such that it can receive at least some of the X-rays emitted by the X-ray source unit.

A single detector cell row usually comprises a large number of detector cells, which are arranged in rows, for example in a ring-segment-shaped or ring-shaped manner. Here, each detector cell typically has one detector pixel, alternatively several detector pixels. A detector pixel has in particular an X-ray-sensitive surface. The X-ray-sensitive surface of all detector pixels of the several detector cell rows form in particular the pixelated detector surface.

In order to ensure spatial coverage in the longitudinal direction of the tunnel, the X-ray detector unit usually has the several detector cell rows. Here, the smaller the respective detector cells are, the more detector cell rows the X-ray detector unit can have.

The scatter radiation shield is embodied in particular to reduce the X-ray radiation. The X-ray-absorbing material can have in particular lead or tungsten, alternatively an alloy of lead and/or tungsten and/or another metal. Other metals are in particular tantalum, rhenium, osmium, iridium, bismuth, platinum, thallium, mercury or gold. The X-ray-absorbing material can also have a carrier material, for example made from an X-ray-transparent material, in particular in the form of a carrier matrix, wherein lead or tungsten, alternatively an alloy of lead and/or tungsten and/or another material, for example in powder form, is embedded in the carrier material. The carrier material can preferably serve exclusively to make the absorber elements mechanically strong and/or stable. The carrier material advantageously does not reduce the absorption properties of the X-ray-absorbing material at all, in particular does so only to a limited extent.

According to one or more example embodiments of the present invention, the several absorber elements are designed exclusively as absorber rings. An absorber ring differs from the prior art in particular such that an absorber element is embodied to be ring-shaped and is arranged in particular about the tunnel. In particular, the scatter radiation shield is not embodied in a grid-like manner. In particular, no absorber element of the scatter radiation shield is aligned parallel to the longitudinal direction of the tunnel. In particular, each absorber element has only one longitudinal extension in the circumferential direction of the tunnel. Depending on their design, absorber elements in accordance with one or more example embodiments of the present invention can have an extension in the longitudinal direction of the tunnel on account of their width. Longitudinal extension is defined here as the longest extension of the absorber element. Advantageously, the length of an absorber element is multiple times greater than its width or its thickness.

The scatter radiation shield is embodied to be essentially ring-shaped on account of the absorber rings. The scatter radiation shield is typically arranged between the X-ray source unit and the X-ray detector unit such that X-ray radiation is absorbed at least partially by the scatter radiation shield before striking the detector surface. The scatter radiation shield thus usually reduces the free inner diameter of the X-ray detector unit.

An advantage of the computed tomography facility is that, on account of the absorber rings, scatter radiation can be at least partially reduced in a static computed tomography facility, wherein simultaneously sufficient X-rays can strike the detector pixels of the X-ray detector unit. One or more example embodiments of the present invention thus increase the image quality in static computed tomography facilities.

In one embodiment, provision is made for the absorber rings to be embodied in the shape of a truncated cone lateral surface and to be rotationally symmetrical. This embodiment is advantageous in particular because it enables the scatter radiation shield to be aligned in the direction of the X-ray source unit. The absorber rings in the shape of a truncated cone lateral surface are embodied in particular to be conical. If the circular arc length of the ring is less than 360°, such an absorber ring can nevertheless have the shape of a truncated cone and be rotationally symmetrical by taking into account the residual circular arc length not implemented. In other words, having the shape of a truncated cone and being rotationally symmetrical does not imply that the circular arc length for this exemplary embodiment has to be 360°. An absorber ring having the shape of a truncated cone lateral surface in particular has the shape of a lateral surface of a truncated cone. An absorber ring of this embodiment has in particular an extension parallel to the longitudinal direction of the tunnel, irrespective of the thickness of the absorber ring. In other words, such an absorber ring having the shape of a truncated cone lateral surface does not lie exclusively in one plane. The absorber ring being rotationally symmetrical means in particular that the truncated cone is a straight truncated cone. The axis of symmetry is in particular a center axis of the tunnel.

In one embodiment, provision is made for at least two absorber rings to differ from one another with respect to their cone angle. This embodiment is advantageous in particular because it enables the scatter radiation shield to be focused. The scatter radiation shield being focused means in particular that the absorber rings having the shape of a truncated cone lateral surface and being rotationally symmetrical are aligned with a section having an extension greater than zero along the center axis of the tunnel, wherein the extension of this section is less than the distance between these at least two absorber rings. It is conceivable in principle for the absorber rings having the shape of a truncated cone and being rotationally symmetrical to be aligned with just one point of the center axis of the tunnel. On account of the rotational symmetry, it is insignificant here whether the cone angle corresponds to the opening angle of the cone or to an angle between the lateral surface and the base surface, since on account of the geometrical relationship the opening angles differ if the angles between the lateral surface and the base surface differ from one another, and vice versa. The cone angles of the at least two absorber rings differ from one another as soon as their difference amount is greater than zero. The difference amount between the cone angles typically lies in a range greater than 0° and/or less than 90°, in particular greater than or equal to 0.1° and/or less than 45°. The further the distance between the at least two absorber rings in the longitudinal direction of the tunnel, the greater the difference amount usually is. It is conceivable for some or all of the several absorber elements to differ from one another with respect to their cone angle. The cone angle difference between adjacent absorber rings could be constant for absorber elements arranged equidistantly one behind the other.

In the following embodiments, the cone angles are described on the basis of the extension direction of the truncated cone lateral surface. The extension direction designates the lengthening of the lateral surface line of the truncated cone lateral surface of the absorber rings. The extension direction of the truncated cone lateral surface of any given absorber ring varies from point to point of the circular arc length of the absorber ring, at least when considered vectorially. The extension direction is therefore dependent on the starting point on the circular arc length. The extension direction starts at a point of the circular arc length and/or of the absorber ring and extends along the truncated cone lateral surface in the direction of the axis of symmetry of the truncated cone. It follows therefrom that all extension directions of the truncated cone lateral surface of a single absorber ring form a spaced-apart circle, depending on the distance from the absorber ring, in the manner of a double cone, said circle delimiting a base surface which lies opposite the base surface of the absorber ring. The distance from the absorber ring is measured along the longitudinal direction of the tunnel and is typically determined as a function of the longitudinal position of the X-ray source unit.

In one embodiment, provision is made for each absorber ring to have such a cone angle that the extension direction of the respective truncated cone lateral surface intersects the ring of several X-ray source points. In this embodiment, the absorber elements are aligned in particular exactly with the X-ray source points. In other words, the spaced-apart circle at the height of the ring of several X-ray source points and the ring of several X-ray source points substantially coincide. The extension direction of the respective truncated cone lateral surface intersecting the ring of several X-ray source points means in particular that preferably each extension direction intersects one of the several X-ray source points or a region of a few centimeters around the one of the several X-ray source points. This surrounding region can have a maximum diameter of 5 cm, preferably maximum 2 cm. Ideally, on account of the scatter radiation shield, each detector cell of the detector surface can detect only X-rays from one X-ray source point of the X-ray source unit. In other words, each detector cell is advantageously aligned with just one X-ray source point of the X-ray source unit. This embodiment is advantageous in particular if the X-ray source point generates the X-rays in the form of a narrow line beam, which can preferably strike the correspondingly aligned detector cell directly. In this embodiment, X-rays which are disposed outside of a narrow line beam are typically absorbed on account of the absorber rings having the shape of a truncated cone lateral surface and the exact alignment thereof with the X-ray source points.

In an alternative embodiment to the afore-cited embodiment, provision is made for each absorber ring to have such a cone angle that the extension direction of the respective truncated cone lateral surface does not intersect the ring of several X-ray source points. This embodiment means in particular that each absorber ring has such a cone angle that the extension direction of the respective truncated cone lateral surface intersects a tunnel section behind the X-ray source unit. In this embodiment, the absorber elements are in particular not aligned exactly with the X-ray source points. In other words, the spaced-apart circle at the height of the ring of several X-ray source points and the ring of several X-ray source points do not coincide. The spaced-apart circle at the height of the ring of several X-ray source points in particular has a significantly smaller diameter with respect to the ring of several X-ray source points. The significantly smaller diameter of the spaced-apart circle is in particular at least 2 cm, for example at least 5 cm, smaller. In other words, it is typically the case in this embodiment that X-rays which are disposed outside of a narrow line beam are typically not absorbed on account of the absorber rings with the shape of a truncated cone lateral surface and the non-exact alignment thereof with the X-ray source points, but instead usually at least two detector cells spaced apart from one another in the circumferential direction of the detector cells are aligned exactly with this X-ray source point.

Provision is made in a development of the afore-cited embodiment for the cone angles to be embodied such that the extension directions form at least one spaced-apart circle at the height of the ring of several X-ray source points, wherein the diameter of each spaced-apart circle differs from the diameter of the ring of several X-ray source points by a factor of less than or equal to 0.98, preferably less than or equal to 0.93. In particular, the diameter of a spaced-apart circle can differ from the diameter of the ring of several X-ray source points by a factor of greater than or equal to 0.15, preferably greater than or equal to 0.5 or greater than or equal to 0.75. Preferably, the diameter of each spaced-apart circle can differ from the diameter of the ring of several X-ray source points by such a factor that, for each X-ray source point, usually up to 180°, in particular between 90° and 150°, preferably substantially 120°, of the ring-shaped detector surface is irradiated in the circumferential direction. This embodiment is advantageous in particular because, for each X-ray source point, usually up to 180°, in particular between 90° and 150°, preferably 120°, of the ring-shaped detector surface is irradiated in the circumferential direction.

Provision is made in one embodiment for the ring-shaped truncated cone lateral surface section at the height of the base surface of at least one absorber ring to be arranged between two adjacent detector cell rows of the detector surface. This embodiment means in particular that the base surface of the absorber ring in the longitudinal direction of the tunnel is arranged not at the same height as one of the two detector cell rows but instead between the two detector cell rows. This embodiment is advantageous in particular because, as a result, the detector pixels of these two detector cell rows are shaded as little as possible by the absorber ring.

In an alternative or additional embodiment to the afore-cited embodiment, provision is made for the ring-shaped truncated cone lateral surface section at the height of the base surface of at least one absorber ring to be arranged above a detector cell row of the detector surface. This embodiment means in particular that the base surface of the absorber ring in the longitudinal direction of the tunnel is arranged substantially at the same height as one detector cell row, in particular not between two detector cell rows. This embodiment is advantageous in particular if this absorber ring has a comparatively low thickness, for example on account of its additive manufacturing.

Provision is made in one embodiment for at least one absorber ring to have a width of between 5 and 50 mm, preferably 20 mm, and/or a thickness of between 2 and 40 μm, preferably 10 μm, and/or a diameter of between 20 cm and/or 200 cm, preferably 80 cm. All absorber rings typically have the same thickness and/or the same width and/or the same diameter.

Provision is made in one embodiment for at least one absorber ring to consist of several absorber ring segments, wherein the several absorber ring segments in each case cover a circular arc with a maximum length of 180°. The several absorber ring segments can be combined in particular in a ring-shaped manner in order to form the at least one absorber ring. It is conceivable in principle for all absorber rings to consist in each case of several absorber ring segments. Here, an absorber ring segment is embodied in principle in the same manner as an absorber ring, but having a shorter circular arc length in comparison with the assembled absorber ring. The absorber ring can be assembled by connecting adjacent absorber ring segments directly in pairs using a fastening mechanism, device and/or means and/or via a carrier structure of the scatter radiation shield. This embodiment is advantageous in particular because, as a result, the at least one absorber ring is easier to manufacture and/or requires less installation space prior to assembly. Furthermore, a single defective absorber ring segment can be replaced more easily in comparison with a one-piece absorber ring.

Provision is made in one embodiment for carrier structures of the scatter radiation shield to be arranged in at least one gap volume, which is delimited by two adjacent absorber rings, in order to fix the adjacent absorber rings relative to one another. The carrier structures can be embodied to fix the two adjacent absorber rings and/or the scatter radiation shield on the gantry and/or on the X-ray detector unit. The fixing can comprise a releasable or non-releasable fastening. The gap volume is typically delimited in the longitudinal direction of the tunnel by the two adjacent absorber rings and/or at right angles to the longitudinal direction of the tunnel by the inner diameter and outer diameter of the adjacent absorber rings. The gap volume can in particular have the shape of a truncated cone lateral surface and/or be rotationally symmetrical. The carrier structures are preferably made from an X-ray-transparent material and/or are embodied to be as X-ray-transparent as possible. It is conceivable for the carrier structures to fill the at least one gap volume only partially with respect to the circumferential direction and/or at right angles to the longitudinal direction of the tunnel, alternatively to fill said gap volume completely. It is possible in principle for carrier structures to be arranged in each case between two adjacent absorber rings. An advantage of carrier structures is that they stabilize the absorber rings.

Provision is made in one embodiment for the carrier structures to be made from plastic. The carrier structures can in particular be made from a matrix consisting of plastic. In particular, the plastic can be polyethylene. Alternatively or additionally, the carrier structures can comprise a foamed material and/or cavities. In comparison with the air pockets in a foam, cavities are spaces enclosed by geometric structures, wherein these geometric structures stabilize the carrier structure itself in the manner of a skeleton. The foamed material and/or cavities advantageously reduce the density of the carrier structures in order to make them even more X-ray-transparent.

Provision is made in one embodiment for the scatter radiation shield, in particular the absorber rings and/or the carrier structures, to be manufactured additively. The additive manufacturing method usually enables the precise manufacturing of complex components, such as for example the scatter radiation shield and/or the carrier structures, by for example applying material additively, in particular layer by layer. The additive manufacturing method can be an additive build-up method, in which the individual layers of the components are applied one after the other. This method can enable a precise control over the component thicknesses and geometries. The material can be applied using various techniques. One possibility can be the selective application of material by an application unit, for example with an application nozzle. Here, the material can be applied in liquid or paste or powder form, in particular in solid form, or can subsequently be cured. Another possibility can be the selective solidification of a powder bed by the application of energy, for example by a laser, for example by selective laser sintering, or another energy source. As a further method, the applied powder can also be cured by way of a chemical process in which additional material is applied, for example by applying, in particular spraying on, a liquid, also known as “binder jetting”. For the application of the material, it is conceivable for one or both of the material compounds to be applied as a liquid. In particular X-ray-transparent plastics, such as for example polyethylene, typically have a relatively low melting point of less than 300° C., while the X-ray-absorbing materials, such as for example tungsten, can have comparatively high melting temperatures in the range of significantly above 1000° C., for example tungsten at 3422° C.

### BRIEF DESCRIPTION OF THE DRAWINGS

The present invention will now be described and explained in greater detail making reference to the exemplary embodiments illustrated in the drawings. In principle, structures and items which remain essentially the same are identified in the following description of the figures with the same reference characters as on the first occurrence of the relevant structure or item.

In the drawings:

FIG. 1 shows first absorber elements from the prior art,

FIG. 2 shows a conventional arrangement of the first absorber elements,

FIG. 3 shows a further view of the conventional arrangement,

FIG. 4 shows second absorber elements from the prior art,

FIG. 5 shows absorber elements according to one or more example embodiments of the present invention,

FIG. 6 shows a computed tomography facility according to one or more example embodiments of the present invention,

FIG. 7 shows a first exemplary embodiment of the computed tomography facility,

FIG. 8 shows a second exemplary embodiment of the computed tomography facility, and

FIG. 9 shows a third exemplary embodiment of the computed tomography facility.

### DETAILED DESCRIPTION

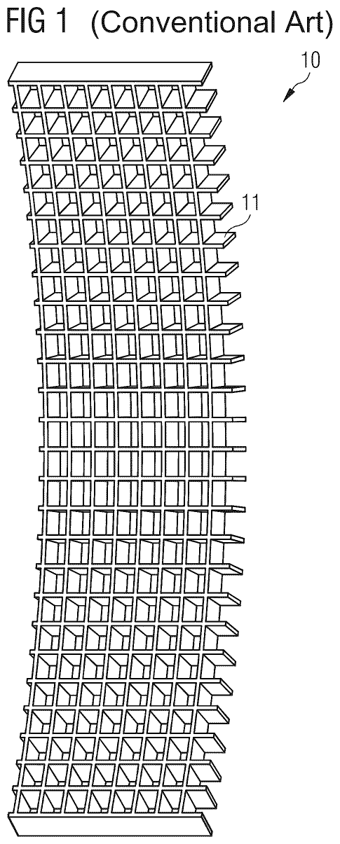

FIG. 1 shows a perspective view of a section of first absorber elements 11 from the prior art. The first absorber elements 11 are embodied in a grid-like manner such that a scatter radiation shield 10 with the first absorber elements 11 has a grid-like shape. The first absorber elements 11 are not embodied exclusively as absorber rings.

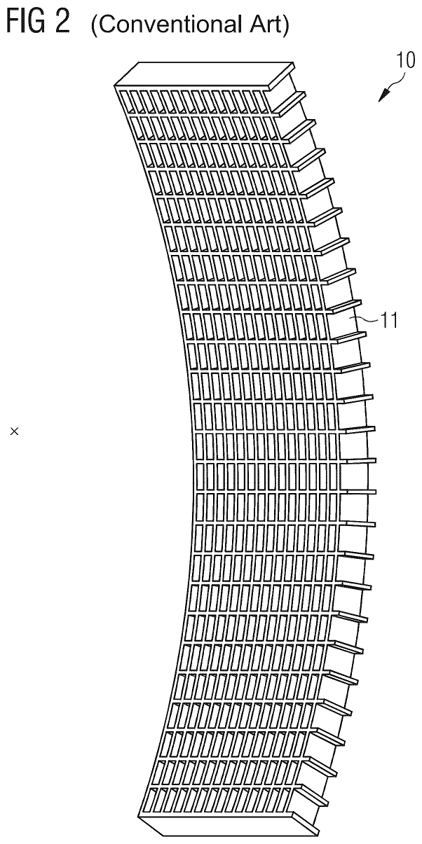

FIG. 2 shows a perspective view of a conventional arrangement of first absorber elements 11 according to FIG. 1 relative to an X-ray source point X in a section.

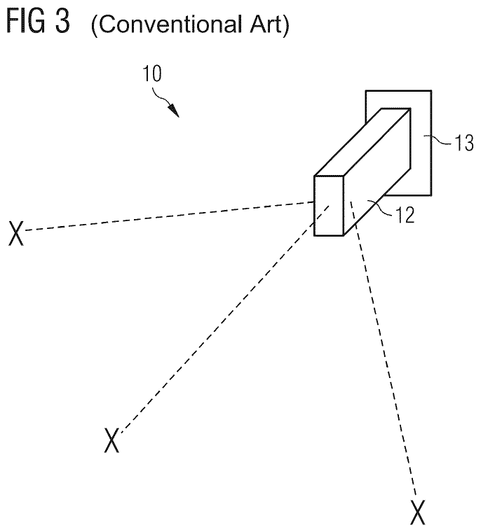

FIG. 3 shows the arrangement according to FIG. 2 in a perspective detailed view. The representation in FIG. 3 is not to scale.

The detailed view shows a shaft 12 with a rectangular cross-section. The shaft 12 typifies the shafts of the scatter radiation shield 10, which are formed by the grid-like first absorber elements 11. Three X-ray source points X for generating X-rays, which are indicated by dashed lines, are drawn in around the shaft 12. The shaft 12 shown in FIG. 3 is aligned with exactly one X-ray source point X, in this case the center of the three shown. X-rays of the center X-ray source point X can strike the exemplary detector pixel 13 and be detected there. The detector pixel 13 is shown as having a greater extension than the clear surface of the shaft 12 purely for illustrative reasons. The surface of a pixel can also be equal to or less than the clear surface of the shaft 12. X-rays of the two X-ray source points positioned away to the side, on the other hand, are prevented by their angled alignment from passing through the shaft 12 and striking the detector pixel 13, and are absorbed-as is otherwise the case for scatter radiation-by those first absorber elements 11 which delimit the shaft 12.

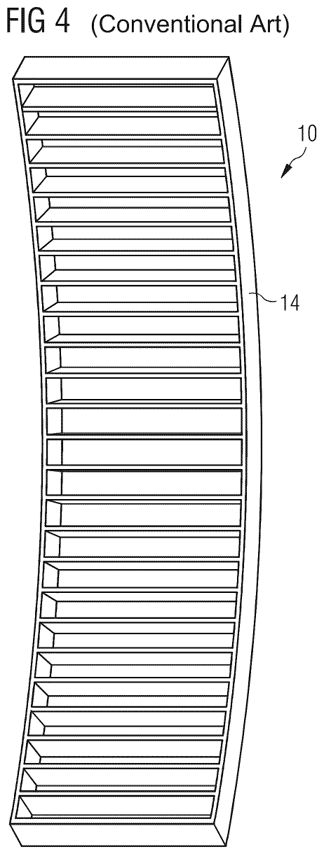

FIG. 4 shows a perspective view of a section of absorber elements 14 from the prior art. The second absorber elements 14 are embodied radially such that the scatter radiation shield 10 is not grid-shaped but has exclusively second absorber elements 14 in the longitudinal direction of the tunnel. The second absorber elements 14 are not embodied exclusively as absorber rings.

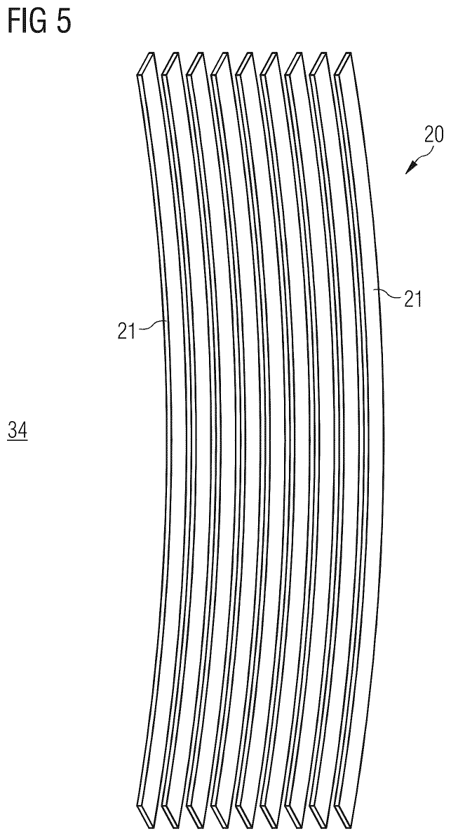

FIG. 5 shows a perspective view of a section of several absorber elements 21 of the scatter radiation shield 20, according to one or more example embodiments of the present invention.

The several absorber elements 21 are made from X-ray-absorbing material. The several absorber elements 21 are designed exclusively as absorber rings. The absorber rings 21 are arranged in a ring-shaped manner about the tunnel 34 and have a fixed position with respect to the gantry 31 (not shown in FIG. 5).

At least one absorber ring has in particular a width of between 5 and 50 mm, preferably 20 mm. Alternatively or additionally, at least one absorber ring can have a thickness of between 2 and 40 μm, preferably 10 μm. Alternatively or additionally, at least one absorber ring can have a diameter of between 20 cm and/or 200 cm, preferably 80 cm.

It is conceivable in principle for at least one absorber ring to consist of several absorber ring segments, wherein the several absorber ring segments in each case cover a circular arc with a maximum length of 180°. The several absorber rings and/or the scatter radiation shield 20 can be manufactured additively.

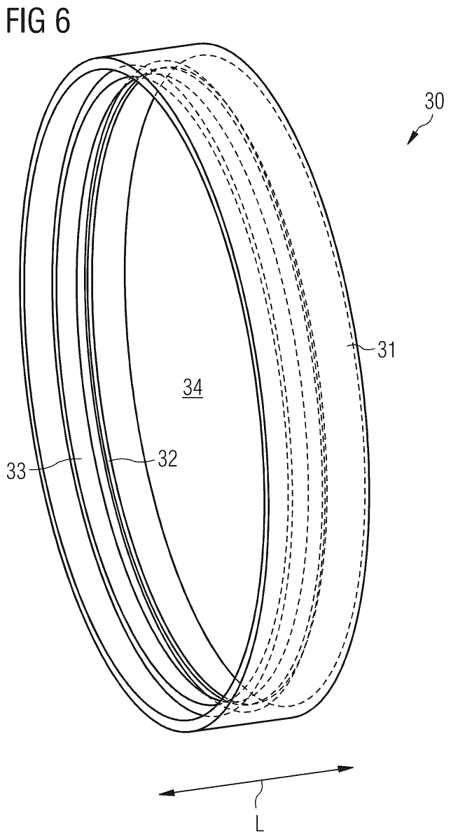

FIG. 6 shows a perspective view of a computed tomography facility 30 according to one or more example embodiments of the present invention.

The computed tomography facility 30 has a gantry 31. The gantry 31 has an X-ray source unit 32, an X-ray detector unit 33 and a tunnel 34. For illustrative reasons, the gantry 31 is shown in FIG. 6 as being transparent in sections and in outline form only.

The X-ray source unit 32 is arranged longitudinally offset relative to the X-ray detector unit 33 with respect to the longitudinal direction L of the tunnel 34. The double arrow L indicates the longitudinal direction of the tunnel 34. The X-ray source unit 32 has several X-ray source points X. The several X-ray source points X are arranged in a ring-shaped manner about the tunnel 34 and in at least one X-ray emitter which has a fixed position with respect to the gantry 31. The several X-ray source points X form part of the X-ray source unit 32 and are not shown separately in FIG. 6. The closed ring shape of the X-ray source unit 32 is intended to indicate the ring on which the several X-ray source points X are arranged, preferably evenly distributed, but not specific positions or a number of X-ray source points X.

The X-ray detector unit 33 has a detector surface. The detector surface is embodied in a ring-shaped manner about the tunnel 34 and has a fixed position with respect to the gantry 31. The X-ray detector unit 33 also has a scatter radiation shield 20. The scatter radiation shield 20 is not shown in FIG. 6 for reasons of clarity, but is embodied in principle in the manner as shown in FIG. 5. The circular arc length of the several absorber elements 21 for the exemplary embodiment in FIG. 6 is 360°.

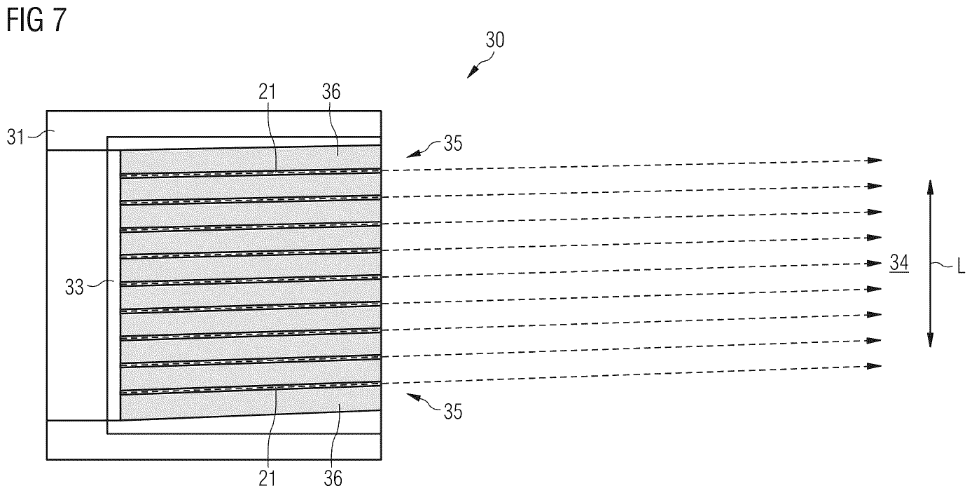

FIG. 7 shows a sectional view of a first exemplary embodiment of the computed tomography facility 30. The sectional plane of FIG. 7 is along the longitudinal direction L of the tunnel 34, which is rotated by 90° in comparison with the previous figure. FIG. 7 shows the section of just one side of the sectioned rings.

FIG. 7 shows nine absorber rings. The number of absorber rings can alternatively be two, three or at least 10 and/or fewer than 100.

The absorber rings, in other words the several absorber elements 21, are embodied to have the shape of a truncated cone lateral surface and to be rotationally symmetrical. The axis of symmetry of the absorber rings is in particular the center axis of the tunnel 34. The dashed arrows indicate the extension direction of the truncated cone lateral surface of the respective absorber ring.

The ring-shaped truncated cone lateral surface section at the height of the base surface of at least one absorber ring can be arranged between two adjacent detector cell rows of the detector surface. The ring-shaped truncated cone lateral surface section at the height of the base surface of all absorber rings can be arranged in each case between two adjacent detector cell rows of the detector surface.

Alternatively, the ring-shaped truncated cone lateral surface section at the height of the base surface of at least one absorber ring can be arranged above a detector cell row of the detector surface. The ring-shaped truncated cone lateral surface section at the height of the base surface of all absorber rings can be arranged in each case above a detector cell row of the detector surface.

The exemplary embodiment in FIG. 7 further shows that carrier structures 36 of the scatter radiation shield 20 are arranged in at least one gap volume 35, which is delimited by two adjacent absorber rings, in order to fix the adjacent absorber rings relative to one another. In this exemplary embodiment, carrier structures 36 are arranged in all gap volumes 35 and completely fill the same. It is alternatively conceivable for carrier structures to fill at least one gap volume only partially. The carrier structures 36 can be made from plastic; alternatively or additionally, they can comprise a foamed material and/or cavities. The carrier structures 36 can be manufactured additively.

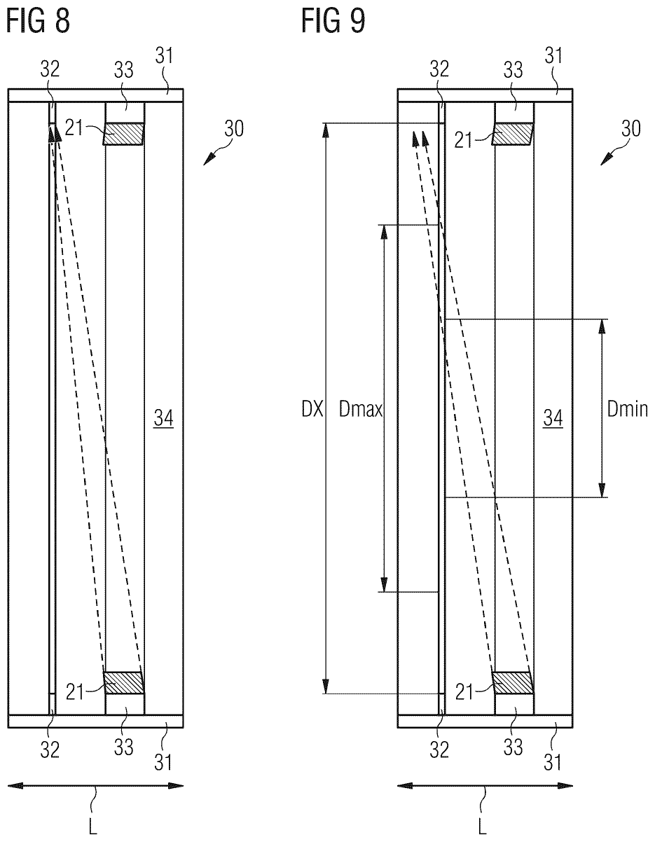

FIG. 8 shows a sectional view of a second exemplary embodiment of the computed tomography facility 30. The sectional plane of FIG. 8 is along the longitudinal direction L of the tunnel 34, which is rotated by 90° in comparison with the previous figure. FIG. 8 shows the section of both sides of the sectioned rings.

The hatching of the absorber elements 21 is not representative of the cone angle. The dashed arrows indicate the extension direction of the truncated cone lateral surface of the respective outermost absorber rings. The extension direction designates the lengthening of the lateral surface line of the truncated cone lateral surface of the absorber rings. At least two absorber rings differ from one another with respect to their cone angle. Each absorber ring has such a cone angle that the extension direction of the respective truncated cone lateral surface intersects the ring of several X-ray source points.

FIG. 9 shows a sectional view of a third exemplary embodiment of the computed tomography facility 30. The sectional plane of FIG. 9 is along the longitudinal direction L of the tunnel 34. FIG. 9 shows the section of both sides of the sectioned rings.

The hatching of the absorber elements 21 is not representative of the cone angle. The dashed arrows indicate the extension direction of the truncated cone lateral surface of the respective outermost absorber rings. At least two absorber rings differ from one another with respect to their cone angle.

Each absorber ring has such a cone angle that the extension direction of the respective truncated cone lateral surface does not intersect the ring of several X-ray source points X. The cone angles are embodied such that the extension directions form at least one spaced-apart circle at the height of the ring of several X-ray source points X, wherein the diameter of each spaced-apart circle Dmin, Dmax differs from the diameter DX of the ring of several X-ray source points X by a factor of less than or equal to 0.98, preferably less than or equal to 0.93. In particular, the diameter Dmin, Dmax of a spaced-apart circle differs from the diameter DX of the ring of several X-ray source points X by a factor of greater than or equal to 0.15, preferably greater than or equal to 0.5 or greater than or equal to 0.75. It is conceivable for the diameter Dmin, Dmax of each spaced-apart circle to differ from the diameter DX of the ring of several X-ray source points X by a factor of greater than or equal to 0.15, preferably greater than or equal to 0.5 or greater than or equal to 0.75.

The diameter of the spaced-apart circles differs as a function of the cone angle of the respective absorber ring. FIG. 9 shows by way of example the diameter DX of the ring of several X-ray source points X, the diameter Dmax of a spaced-apart circle of an absorber ring with the maximum diameter and the diameter Dmin of a spaced-apart circle of an(other) absorber ring with the minimal diameter.

The absorber elements, according to one or more example embodiments of the present invention, are designed exclusively as absorber rings, so that the scatter radiation shield has exclusively absorber elements in the circumferential direction about the tunnel. The absorber elements, according to one or more example embodiments of the present invention, are in particular not embodied in a grid-shaped manner and/or radially.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although one or more example embodiments of the present invention has been illustrated and described in detail by way of the preferred exemplary embodiments, one or more example embodiments of the present invention is nevertheless not restricted by the examples given and other variations can be derived therefrom by a person skilled in the art without departing from the protective scope of one or more example embodiments of the present invention.

Independent of the grammatical term usage, individuals with male, female or other gender identities are included within the term.

What is claimed is:

1. A computed tomography device, comprising:
a gantry having an X-ray source unit, an X-ray detector unit and a tunnel, wherein
the X-ray source unit is arranged longitudinally offset relative to the X-ray detector unit with respect to a longitudinal direction of the tunnel,
the X-ray source unit has a plurality of X-ray source points, which are arranged in a ring-shaped manner about the tunnel and in at least one X-ray emitter, which has a fixed position with respect to the gantry,
the X-ray detector unit has a detector surface, which is embodied in a ring-shaped manner about the tunnel and has a fixed position with respect to the gantry,
the X-ray detector unit has a scatter radiation shield,
the scatter radiation shield has a plurality of absorber elements made from X-ray-absorbing material, and
the plurality of absorber elements are designed exclusively as absorber rings, which are arranged in a ring-shaped manner about the tunnel and have a fixed position with respect to the gantry.

2. The computed tomography device as claimed in claim 1, wherein the absorber rings have a shape of a truncated cone lateral surface and are rotationally symmetrical.

3. The computed tomography device as claimed in claim 2,
wherein at least two absorber rings differ from one another with respect to a cone angle.

4. The computed tomography device as claimed in claim 2, wherein each absorber ring has a cone angle such that an extension direction of a respective truncated cone lateral surface intersects a ring of several X-ray source points.

5. The computed tomography device as claimed in claim 2, wherein
each absorber ring has a cone angle such that an extension direction of a respective truncated cone lateral surface does not intersect a ring of several X-ray source points,
cone angles are configured such that the extension directions form at least one spaced-apart circle at a height of the ring of several X-ray source points, and
a diameter of each spaced-apart circle differs from a diameter of the ring of several X-ray source points by a factor of less than or equal to 0.98.

6. The computed tomography device as claimed in claim 5,
wherein the diameter of a spaced-apart circle differs from the diameter of the ring of several X-ray source points by a factor of greater than or equal to 0.15.

7. The computed tomography device as claimed in claim 2,
wherein a ring-shaped truncated cone lateral surface section at a height of a base surface of at least one absorber ring is arranged between two adjacent detector cell rows of the detector surface.

8. The computed tomography device as claimed in claim 2, wherein a ring-shaped truncated cone lateral surface section at a height of a base surface of at least one absorber ring is arranged above a detector cell row of the detector surface.

9. The computed tomography device as claimed in claim 1, wherein at least one absorber ring has at least one of a width of between 5 and 50 mm, a thickness of between 2 and 40 μm, or a diameter of between 20 cm and 200 cm.

10. The computed tomography device as claimed in claim 1, wherein at least one absorber ring includes a plurality of absorber ring segments, and wherein each of the plurality of absorber ring segments covers a circular arc with a maximum length of 180°.

11. The computed tomography device as claimed in claim 1, wherein carrier structures of the scatter radiation shield are arranged in at least one gap volume, and wherein the at least one gap volume is delimited by two adjacent absorber rings, in order to fix the two adjacent absorber rings relative to one another.

12. The computed tomography device as claimed in claim 11,
wherein the carrier structures are plastic.

13. The computed tomography device as claimed in claim 11,
wherein the carrier structures comprise at least one of a foamed material or cavities.

14. The computed tomography device as claimed in claim 11, wherein the carrier structures completely fill the at least one gap volume.

15. The computed tomography device as claimed in claim 1, wherein the scatter radiation shield is manufactured additively.

16. The computed tomography device as claimed in claim 5, wherein the diameter of each spaced-apart circle differs from the diameter of the ring of several X-ray source points by a factor of less than or equal to 0.93.

17. The computed tomography device as claimed in claim 6, wherein the diameter of the spaced-apart circle differs from the diameter of the ring of several X-ray source points by a factor of greater than or equal to 0.5.

18. The computed tomography device as claimed in claim 1, wherein at least one absorber ring has at least one of a width of 20 mm, a thickness of 10 μm, or a diameter of between 20 cm and 200.

19. The computed tomography device as claimed in claim 15, wherein at least one of the absorber rings or carrier structures of the scatter radiation shield are manufactured additively.

20. The computed tomography device as claimed in claim 3, wherein
each absorber ring has a cone angle such that an extension direction of a respective truncated cone lateral surface does not intersect a ring of several X-ray source points,
cone angles are configured such that the extension directions form at least one spaced-apart circle at a height of the ring of several X-ray source points, and
a diameter of each spaced-apart circle differs from a diameter of the ring of several X-ray source points by a factor of less than or equal to 0.98.
